(19) United States
(12) Patent Application Publication
(10) Pub. No.: US 2005/0255169 A1
(43) Pub. Date: Nov. 17, 2005

(54) Adjuvant containing xenon

(75) Inventors: Carsten Pilger, Rheurdt (DE); Peter Neu, Duisburg (DE); Matthias Reyle-Hahn, Berlin (DE)

Correspondence Address: CONNOLLY BOVE LODGE & HUTZ, LLP, P O BOX 2207, WILMINGTON, DE, 19899, US

(73) Assignee: MESSER GRIESHEIM

(21) Appl. No.: 10/518,067

(22) PCT Filed: Jul. 4, 2003
(86) PCT No.: PCT/EP03/07186

§ 371 Date: Dec. 13, 2004

(30) Foreign Application Priority Data
Jul. 5, 2002 (DE) 102 30 544.7
Aug. 10, 2002 (DE) 102 36 762.0
Aug. 10, 2002 (DE) 102 36 761.2
Aug. 10, 2002 (DE) 102 36 760.4

(51) Int. Cl. A61K 33/00; A61K 31/46; A61K 31/24

(52) U.S. Cl. 424/600; 514/304; 514/537

(57) ABSTRACT

The invention relates to xenon or xenon-containing gases which is/are used for producing an adjuvant that is administered together with another medicament, such as an antiviral, antibacterial, antimycotic, neuroprotective, or anticancerogenic agent, a parasympathomimetic, parasympatholytic, spasmolytic, symnpathomimetic, sympatholytic, preceptor blocker, tranquilizer, neuroleptic, antidepressant, analgesic, antipyretic, anticephalalgic, anti-Parkinson's drug, analeptic, antiepileptic, antiemetic, emetic, a substance influencing coagulation, an amino acid, a vitamin, or a hormone.

The invention relates to a medicament comprising xenon.

WO 02/22141 A2 describes the use of xenon or xenon-containing gases as medicaments, in particular cardiovascular agents.

DE 19933704 A1 describes the use of a liquid preparation which comprises a lipophilic gas such as xenon for neuroprotection and neuroregeneration.

Many pharmacological active ingredients reach the target site (site of action) in a patient's body via the bloodstream. If the blood flow in a part of the body is restricted, the active ingredients cannot reach the site of action in sufficient quantity. Bloodlessness of individual parts of organs as a result of insufficient blood supply is referred to as ischemia. Ischemia arises for example in connection with thrombosis or embolism. Particularly serious impairments of blood flow in the brain occur in connection with stroke.

For many medicaments an inadequate concentration of active ingredient in the brain also derives from the blood-brain barrier. Medicaments are administered in higher dose for this reason.

Means assisting the effect of a medicament are referred to in medicine as adjuvant.

The invention is based on the object of improving the medicament supply or active ingredient supply to parts of the body, in particular of the brain.

The invention relates to an adjuvant having the features described in claim 1.

Xenon or xenon-containing gases (gas mixtures) are used as adjuvant or as component of an adjuvant.

The adjuvant assists in particular medicaments whose active ingredient or active ingredients are transported via the bloodstream. Medicaments with one or more active ingredients which are transported via the bloodstream are referred to herein as hemogenous medicaments. Medicaments assisted by the adjuvant are preferably medicaments intended to act in the brain (cerebral medicaments), especially hemogenous cerebral medicaments.

The adjuvant is preferably administered by inhalation. The adjuvant is therefore preferably employed as inhalable medicament.

Adjuvant and supported medicament which are employed together are regarded as combination product or combination medicament, the adjuvant and supported medicament being administered together in one medicament (the adjuvant is present in the medicament) or as separate medicaments.

The combination medicament composed of a xenon-containing medicament, the adjuvant and a further medicament (the medicament assisted by the adjuvant) serve for simultaneous, separate or sequential use of the medicaments (of the components of the combination medicament). The combination medicament preferably consists of an inhalable medicament which comprises xenon (e.g. xenon or a xenon-containing gas), and of a hemogenous medicament, e.g. a medicament which is administered orally or parenterally. The adjuvant or inhalable medicament comprises gaseous xenon in pharmacologically or therapeutically effective amount, in particular in an amount, concentration or dosage which has a subsedative effect, sedative effect, subanalgesic activity, analgesic activity, subhypnotic activity, hypnotic activity, subanesthetic activity or anesthetic activity.

Subanesthetic amounts of xenon mean the amounts, concentrations or dosages of xenon which are insufficient for general anesthesia. Subsedative amounts of xenon mean the amounts, concentrations or dosages of xenon which are insufficient for sedation. Subhypnotic amounts of xenon mean the amounts, concentrations or dosages of xenon which are insufficient for inducing and maintaining sleep. Subanalgesic or analegesically active amounts of xenon mean the amounts, concentrations or dosages of xenon which are insufficient for an analgesic effect.

The combination medicament is usually employed for humans or mammals.

The medicaments which are combined with the adjuvant include besides the medicaments with pharmacologically active substances (active ingredients) also diagnostic aids, x-ray contrast agents, radioactive isotopes.

The adjuvant is used for example in combination with an antiviral, antibacterial, antimycotic, neuroprotective, anticarcinogenic, sedative, analgesically or anesthetically acting substance, in particular with opioids (e.g. sufentanil, remifentanil), anesthetics, volatile anesthetics (e.g. methoxyflurane, halothane, enflurane, isoflurane, sevoflurane and desflurane), α2-adrenoceptor agonists (e.g. clonidine, dexmedetomidine) or catecholamines. The active ingredients of the assisted medicaments are usually organic substances.

The adjuvant is advantageously combined with parasympathomimetics, parasympatholytics, spasmolytics, sympathomimetics, sympatholytics, β-receptor blockers, tranquilizers, neuroleptics, antidepressants, sedatives (sedating agents), analgesics, antipyretics, migraine remedies, antiparkinson agents, analeptics, antiepileptics, antiemetics, emetics, substances influencing blood clotting, amino acids, vitamins or hormones.

The adjuvant is further employed with medicaments for NOS inhibition, with medicaments for treating migraine, with medicaments for treating septic shock, multiple sclerosis, inflammations or inflammatory pain.

The adjuvant is used for example with medicaments for the treatment and/or prophylaxis of stroke, reperfusion damage, brain trauma, of impairments of the blood flow in the brain, of impairment of cerebral perfusion, of cognitive disorders or of post-ischemia syndrome.

The adjuvant is further employed with a medicament for neuroprotection, a medicament for the prophylaxis and/or therapy of impairments of cognitive performance, a medicament for improving the oxygen supply in the brain or a medicament for promoting blood flow in the brain.

The combination medicaments include in particular the adjuvant and a medicament for the therapy of disorders associated with a loss of cognitive and memory functions, e.g. in the course of pathological aging process such as, for example, Parkinson's disease, Alzheimer's disease, the organic brain syndrome, AIDS dementia, depressive pseudodementias, dementing syndromes, deliria as acute organic brain syndromes, intoxications, withdrawal syndromes or cytopathic influences.

The adjuvant is advantageously employed in combination with medicaments for chronic neurodegenerative disorders such as Huntington's disease, amyotropically lateral sclerosis, Parkinson's disease, AIDS dementia, Alzheimer's disease or acute neurodegenerative disorders such as ischemias of the brain and neurotraumata.

The adjuvant is advantageously used in combination with sedative substances, in particular with centrally sedative substances. The sedative substances are usually organic active ingredients having a sedative effect. The sedative substances are usually contained in a medicament (sedative medicament, sedating agent or sedative) which is administered with the adjuvant, in particular as combination product or combination medicament. Such combination products or combination medicaments therefore generally consist of a xenon-containing medicament as adjuvant and of a sedative medicament for simultaneous, separate or sequential use of the medicaments. Such a combination medicament preferably consists of an inhalable medicament with xenon or a xenon-containing gas and a sedative medicament which is administered orally or parenterally. The adjuvant or inhalable medicament is administered for example in an amount, concentration or dosage with a subsedative or sedative effect.

Sedative medicaments or active ingredients (sedatives) are, for example, long-acting barbiturates such as barbital or phenobarbital, medium- and short-acting barbiturates such as allobarbital, amobarbital, aprobarbital, brallobarbital, cyclobarbital, pentobarbital, proallylanol, secobarbital and vinylbital, alcohols and aldehydes such as chloral hydrate, methylpentynol and paraldehyde and benzodiazepines such as alprazolam, bromazepam, brotizolam, diazepam, flunitrazepam, flurazepam, loprazolam, lormetazepam, midazolam, nitrazepam, oxazepam, temazepam and triazolam.

The adjuvant effects a potentiation of known sedatives. Conventional sedatives can thus be administered in lower dose, whereby side effects can be considerably reduced or avoided.

The adjuvant is further employed in combination with an analgesic. The combination product or combination medicament generally consists of a xenon-containing medicament and an analgesic for simultaneous, separate or sequential use, in particular for the treatment and prophylaxis of pain. The combination medicament preferably consists of an inhalable medicament with xenon or a xenon-containing gas and an analgesic which can be administered orally or parenterally.

Analgesics or analgesic active ingredients are analgesics with morphine-like effect such as buprenorphine, cetobemidone, codeine, dextromoramide, dextropropoxyphene, fentanyl, hydromorphone, meptazinol, methadone, morphine, nalbuphine, nefopam, opium complete extract, oxycodone, pentazocine, pethidine (meperidine), piritramide, tilidine, tramadol or naloxone. Further analgesics are salicylic acid derivatives, pyrazolone derivatives and aminophenol derivatives.

Salicylic acid derivatives are acetylsalicylic acid, benorilate, diflunisal, ethenzamide gentisate sodium, salacetamide, salicylamide, salicylic acid or salsalate. Pyrazolone derivatives are metamizole (noramidopyrine), morazone, phenazone or propyphenazone. An aminophenol derivative is paracetamol. Further analgesics are quinine, flunixine, flupirtine or benzyl phenylglycolate (mandelic acid benzyl ester, benzyl mandelate).

The adjuvant is further employed in combination with a local anesthetic agent (local anesthetic). Local anesthetic agents are, for example, articaine, benzovaine, bupivacaine, butanilicaine, butoxycaine, cinchocaine, cocaine, etidocaine, fomocaine, lidocaine, mepivacaine, oxetacaine, oxybuprocaine, pramocaine, prilocaine, procaine, proxymetacaine, ropivacaine, tolycaine or tetracaine. The assisted medicament may also be a mixture of two or more local esthetic agents.

The adjuvant is preferably employed as a gas mixture which supports breathing and comprises xenon and oxygen.

The provided adjuvant or the adjuvant produced directly on use, in particular in the immediate vicinity of the patient, is for example a gas mixture which comprises from 1 to 80% by volume (based on standard conditions, i.e. 20° C., 1 bar absolute) xenon (e.g. remainder oxygen). The medicament which is administered to the patient advantageously comprises xenon in subanesthetic amounts. Subanesthetic amounts of xenon mean amounts or concentrations of xenon which are insufficient for anesthesia. These are in general amounts of up to 70% by volume xenon, preferably up to 65% by volume, particularly preferably up to 60% by volume, in particular up to 50% by volume is xenon. Pure xenon is accordingly metered in the stated concentrations into the patient's respiratory gas. This means that the respiratory gas supplied to the patient comprises, for example, from 5 to 60% by volume, 5 to 50% by volume, 5 to 40% by volume, 5 to 30% by volume or 5 to 20% by volume xenon. In special cases, e.g. for prophylaxis, especially during prolonged ventilation, a dosage of xenon in the respiratory gas with a low concentration, for example from 1 to 35% by volume, 5 to 25% by volume or 5 to 20% by volume xenon in the respiratory gas, may be advantageous.

The gaseous adjuvant preferably comprises besides xenon one or more gases or substances which are gaseous at body temperature under atmospheric pressure. Gas mixtures which can be used are, for example, xenon-oxygen gas mixtures or gas mixtures of xenon and one or more inert gases such as nitrogen or a rare gas (e.g. helium) or xenon-oxygen-inert gas gas mixtures. Suitable gas mixtures are described in WO 02/22141 A2, to which reference is hereby made.

Gaseous xenon (pure xenon) is generally provided as compressed gas in compressed gas containers such as compressed gas cylinders or pressurized cans. It is also possible to provide xenon-containing gas mixtures in compressed gas containers. The gaseous medicament can also be provided in a container as liquefied gas or gas mixture or in cryogenically solidified form.

The adjuvant is usually administered using a ventilation machine with a gas-measuring unit or with an anesthesia machine. The adjuvant is advantageously produced directly for use from the pure gases, for example by mixing xenon, oxygen and, where appropriate, an inert gas (e.g. with the aid of an anesthesia machine or a gas-metering device) in the immediate vicinity of the patient.

One, more than one or all of the gas components of the gaseous adjuvant, in particular xenon and oxygen or a respiratory gas are advantageously mixed with the aid of a gas-metering device. The gas-metering device is used to vary the concentrations of the gas components advantageously during a ventilation. The device and the various methods for gas metering, in particular continuous and discontinuous gas metering with constant or variable gas component concentration, are described in DE 197 46 742 A1 and WO 98/31282, to which reference is hereby made.

The adjuvant or combination medicament is also administered for example with a heart-lung machine.

The combination medicament is employed in the following way, for example. Initially a xenon-containing gas is administered as adjuvant in a subanesthetically and/or sedatively active amount. In a next phase of the sequential administration of the medicament, the hemogenous medicament is administered.

The adjuvant is usually administered as dry, moist gas or water vapor-saturated gas to the patient.

The adjuvant is, for example, also a liquid preparation comprising xenon. Such a liquid preparation is described in DE 19933704 A1 to which reference is hereby made.

1. An adjuvant comprising xenon or a xenon-containing gas.

2. The adjuvant as claimed in claim 1, characterized in that the adjuvant is an adjuvant for hemogenous medicaments or active ingredients.

3. The adjuvant as claimed in claim 2, characterized in that the adjuvant is gaseous.

4. The adjuvant as claimed in claim 3, characterized in that the adjuvant is administered by inhalation.

5. The adjuvant as claimed in claim 4, characterized in that xenon is present in a pharmacologically effective amount, in particular in an amount having a subsedative effect, sedative effect, subanalgesic activity, analgesic activity, subhypnotic activity, hypnotic activity, subanesthetic activity or anesthetic activity.

6. A combination medicament which comprises xenon or a xenon-containing gas as adjuvant and at least one additional medicament for simultaneous, separate or sequential use of adjuvant and medicament.

7. The combination medicament as claimed in claim 6, characterized in that the additional medicament comprises at least one medicament from the group consisting of medicaments with an antiviral, antibacterial, antimycotic, neuroprotective, anticarcinogenic, sedative, analgesically or anesthetically acting substance; opioids; sufentanil, remifentanil; anesthetics, volatile anesthetics; methoxyflurane, halothane, enflurane, isoflurane, sevoflurane and desflurane; local anesthetics; articaine, benzovaine, bupivacaine, butanilicaine, butoxycaine, cinchocaine, cocaine, etidocaine, fomocaine, lidocaine, mepivacaine, oxetacaine, oxybuprocaine, pramocaine, prilocaine, procaine, proxymetacaine, ropivacaine, tolycaine or tetracaine; 2-adrenoceptor agonists, clonidine, dexmedetomidine; catecholamines, parasympathomimetics, parasympatholytics, spasmolytics, sympathomimetics, sympatholytics, β-receptor blocks, tranquilizers, narcoleptics, antidepressants, sedatives, centrally sedative sedating agents, analgesics, antipyretics, migraine remedies, antiparkinson agents, analeptics, antiepileptics, antiemetics, emetics, substances influencing blood clotting, amino acids, vitamins or hormones; medicament s for NOS inhibition, medicaments for treating migraine, medicaments for treating septic shock, medicaments for treating multiple sclerosis, medicaments for treating inflammations or inflammatory pains; hemogenous cerebral medicaments; medicaments for the treatment and/or prophylaxis of stroke, reperfusion damage, brain trauma, of impairments of blood flow in the brain, of impairment of cerebral perfusion, of cognitive impairments or of postischemia syndrome; barbiturates; barbital or phenobarbital, allobarbital, amobarbital, aprobarbital, brallobarbital, cyclobarbital, pentobarbital, proallylanol, secobarbital and vinylbital, chloral hydrate, methylpentynol, paraldehyde; benzodiazepines, alprazolam, bromazepam, brotizolam, diazepam, flunitrazepam, flurazepam, loprazolam, lormetazepam, midazolam, nitrazepam, oxazepam, temazepam and triazolam; medicaments for neuroprotection, medicaments for the prophylaxis and/or therapy of impairments of cognitive performance; medicaments for Parkinson's disease, Alzheimer's disease, organic brain syndrome, AIDS dementia, depressive pseudodementias, dementing syndromes, deliria as acute organic brain syndromes, intoxications, withdrawal syndromes or cytopathic influences; medicaments for chronic neurodegenerative disorders; medicaments for Huntington's disease, amyotropically lateral sclerosis, Parkinson's disease, AIDS dementia, Alzheimer's disease or acute neurodegenerative disorders; medicaments for ischemias of the brain or neurotraumata; diagnostic aids, x-ray contrast agents or radioactive isotopes.

8. The use of xenon, of a xenon-containing gas or of a xenon-containing preparation as adjuvant or to produce an adjuvant, in particular as adjuvant or to produce an adjuvant for hemogenous medicaments or for brain-penetrating medicaments or active ingredients.

9. The use as claimed in claim 8, characterized in that the adjuvant is administered with at least one medicament for the treatment of acute and chronic cerebral disorders or impairments or a medicament for the treatment and/or prophylaxis of ischemic brain disorders or the sequelae of a cerebral ischemia or the adjuvant is administered with at least one medicament for the treatment and/or prophylaxis of stroke, reperfusion damage or brain trauma.

10. The adjuvant as claimed in claim 1, characterized in that the adjuvant is gaseous.

11. The adjuvant as claimed in claim 1, characterized in that the adjuvant is administered by inhalation.

12. The adjuvant as claimed in claim 1, characterized in that xenon is present in a pharmacologically effective amount, in particular in an amount having a subsedative effect, sedative effect, subanalgesic activity, analgesic activity, subhypnotic activity, hypnotic activity, subanesthetic activity or anesthetic activity.
